SEQUENCE LISTING (3 sequences)

SEQ ID NO: 1 - protein, 11 aa, Artificial Sequence
  polyhistidine tag
MKKGHHHHHHG

SEQ ID NO: 2 - protein, 240 aa, Artificial Sequence
  ERRy ligand binding domain with polyhistidine tag
MKKGHHHHHHGPAKKPYNKIVSHLLVAEPEKIYAMPDPTVPDSDIKALTTLCDLADRELVVIIGWAKHIPGFSTLSLADQMSLLQSAWMEILILGVVYRSLSFEDELVYADDYIMDEDQSKLAGLLDLNNAILQLVKKYKSMKLEKEEFVTLKAIALANSDMHIEDVEAVQKLQDVLHEALQDYEAGQHMEDPRRAGKMLMTLPLLRQTSTKAVQHFYNIKLEGKVPMHKLFLEMLEAKV

SEQ ID NO: 3 - protein, 25 aa, Artificial Sequence
  modified streptavidin
LERNNIKQAANNSLLLHLLKSQTIP

(19) United States
(12) Patent Application Publication
(10) Pub. No.: US 2005/0074765 A1
(43) Pub. Date: Apr. 7, 2005

(54) Methods for identifying and using modulators of estrogen related receptor gamma

(76) Inventors: Steven G. Blanchard, Durham, NC (US); Aaron B. Miller, Durham, NC (US); Lisa A Orband-Miller, Durham, NC (US)

Correspondence Address: DAVID J LEVY, CORPORATE INTELLECTUAL PROPERTY;GLAXOSMITHKLINE, FIVE MOORE DR., PO BOX 13398, RESEARCH TRIANGLE PARK, NC, 27709-3398, US

(21) Appl. No.: 10/491,677

(22) PCT Filed: Oct. 16, 2002
(86) PCT No.: PCT/US02/32917

(51) Int. Cl. C12Q 1/68; G06F 19/00; G01N 33/48; G01N 33/50

(52) U.S. Cl. 435/6; 702/20

(57) ABSTRACT

Methods of identifying compounds that interact with ERRγ and are useful in the treatment of ERRγ- and ER-mediated diseases, risk factors, and conditions are provided. Pharmaceutical compositions containing compounds that interact directly with ERRγ and methods of using these compositions in the treatment of ERRγ- and ER-mediated diseases, risk factors, and conditions are also provided.

### FIELD OF THE INVENTION

The present invention relates to compounds and methods for identifying compounds that interact directly with Estrogen Related Receptor γ (ERRγ), and are useful for the treatment of ERRγ-mediated diseases, risk factors, or conditions including, but not limited to, osteoporosis, cancer, menopause, and cardiovascular disease. These compounds can also affect the estrogen receptor signaling system, and are useful for the treatment of Estrogen Receptor (ER)-mediated diseases, risk factors, and conditions including, but not limited to, osteoporosis, cancer, menopause, and cardiovascular disease.

### BACKGROUND OF THE INVENTION

The Estrogen Related Receptor garmma (ERRγ:NR3B3 is an orphan nuclear receptor. ERRγ is the third orphan nuclear receptor in the Estrogen Related Receptor family to be cloned. There are two other receptors in the ERR subfamily, ERRα and ERRβ. The sequence identity in the ligand binding domain of ERRγ and ERRβ is 77% (Johnson et. al. Mol. Endo. 1997 11:342-52). ERRγ was found by using glucocorticoid receptor interacting protein 1 (GRIP1) as bait in the yeast two-hybrid system.

ERRγ is highly expressed in adult tissue such as the brain, skeletal muscle, heart, kidney, and retina. It is also expressed in fetal tissue such as the placenta, brain, heart, skeletal muscle, kidney and lung (Bonnelye et al. Mol. Endo. 1997 11:905-16).

Receptors of the ERR subfamily exhibit 68% amino acid identity to estrogen receptor α (ERα) in the DNA binding domain and 36% identity to ERα in the Ligand Binding Domain. Thus, receptors of the ERR subfamily are able to bind to the estrogen response element (ERE). However despite the similarities between ERRs and ERs, to date there is no detectable binding of estradiol or steroid like estrogen compounds to ERRγ. Further, the overall distribution of ERRγ expressed in the mouse brain indicates functional differences between ERRγ and the ERs (Lorke et al. Molecular Brain Research 2000 77:277-280).

While a large amount of work at the molecular level has been performed comparing the ERRs to themselves and to the ER (Heard et. al. Mol. Endo. 2000 14:382-92; Bonnelye et. al. Mol. Endo. 1997 11:905-16; Johnston et. al. Mol. Endo. 1997 11:342-52; Vanacker et. al. Mol. Endo. 1999 13:764-73), little biochemical information is available on ligands of ERRs, and in particular ERRγ.

Two organochlorine pesticides have been reported as antagonists for ERRα in a yeast based assay (Yang et al. Cancer Research 59 (1999) 4519-4524).

Diethylstilbestrol (DES) has been reported to regulate trophoblast stem cell differentiation as a ligand for ERRβ (Tremblay et al. Genes & Development 2001 15:333-338). In these studies, DES was demonstrated to interact with ERRα, ERRβ, and ERRγ to suppress coactivator binding and transcription from a reporter gene in a nuclear receptor-coactivator interaction assay using fluorescent resonance emission transfer (FRET; Zhou et al. Mol. Endocrinol. 1998 12:1594-1604).

PCT/JP99/06097 discloses a 458 amino acid protein referred to as ERRγ obtained by cloning a human brain-origin cDNA library and a gene of 1377 bp encoding the same.

The present invention provides methods for identifying compounds which interact with ERRγ. Compositions identified via the methods of the present invention are believed to be useful in the treatment of diseases and conditions such as osteoporosis, cancer, menopause and cardiovascular disease.

### SUMMARY OF THE INVENTION

An object of the present invention is to provide methods for identifying compounds useful in the treatment of osteoporosis, cancer, menopause, and cardiovascular disease comprising the step of determining whether the compound interacts with ERRγ.

Another object of the present invention is to provide methods for treating osteoporosis, cancer, menopause cardiovascular disease and other conditions or disorders mediated by ERRγ or ER via administration of a compound which interacts with ERRγ.

Yet another object of the present invention is to provide compositions for use in the treatment of osteoporosis, cancer, menopause, cardiovascular disease and other conditions or disorders mediated by ERRγ or ER which comprise a compound which interacts with ERRγ and one or more pharmaceutically acceptable carriers or excipients.

### DETAILED DESCRIPTION OF THE INVENTION

Estrogen Related Receptor Gamma (ERRγ) is a member of the Nuclear Receptor family of transcription regulatoins, of which ERRα and ERRβ are close relatives. In humans, ERRγ exists as three distinct splice variants with different tissue distributions. ERRγ1 is expressed in brain, placenta, heart, retina. ERRγ2 is expressed in kidney and pancreas. ERRγ3 is expressed in skeletal muscle only, Heard et al. Mol. Endo. 2000 14:382-392). The ligand-binding domain of all three ERRγ splice variants is identical so compounds which bind selectively to the LBD of ERRγ will selectively regulate the activity of ERRγ over the other ERR subtypes.

The present invention relates to compounds and methods for identifying compounds useful in the treatment of osteoporosis, cancer, menopause, and cardiovascular disease which interact with ERRγ. These compounds are also referred to herein as ligands of ERRγ. By “interact” for purposes of the present invention, it is meant that the compound binds with this receptor. More preferably it is meant that the compound binds with and modulates ERRγ expression and/or activity. It is preferred that the compound binds directly with the receptor at its ligand binding domain, thereby inhibiting the ability of the receptor to biind with other ligands. By “modulate”, “modulates” or “modulating” it is meant that expression of ERRγ is upregulated or downregulated and/or that activity of ERRγ is increased or decreased. Compounds which upregulate expression and/or increase activity of ERRγ are referred to as ERRγ agonists while compounds which downregulate expression and/or decrease activity of ERRγ are referred to as ERRγ antagonists.

The present invention also relates to methods for using compounds which interact directly with ERRγ in the treatment of ERRγ mediated diseases, risk factors, or conditions. Exemplary ERRγ mediated diseases, risk factors, and conditions include, but are not limited to, osteoporosis, cancer, menopause, and cardiovascular disease. Further, as will be understood by one of skill in the art upon reading this disclosure, additional ERRγ mediated diseases, risk factors, or conditions can be identified based upon tissue distribution of this nuclear receptor as well as current therapies for known compounds which can now be identified in accordance with the present invention as interacting with ERRγ. The compounds of the present invention can also affect the estrogen receptor (ER) signaling system. Thus, the present invention also relates to methods for the treatment of ER-mediated diseases, risk factors, and conditions including, but not limited to, osteoporosis, cancer, menopause, and cardiovascular disease.

To identify compounds which interact directly with ERRγ, also referred to herein as ERRγ ligands, a fluorescence resonance emission transfer (FRET) assay (Parks et al. Science 1999 284:1365-1368) for ERRγ was developed. FRET assays comprise the steps of exposing a sample portion comprising the donor located at a first position and the acceptor located at the second position to light at a first wavelength capable of inducing a first electronic transition in the donor, wherein the donor comprises a complex of lanthanide chelate and a lanthanide capable of binding the chelate and wherein the spectral overlap of the donor emission and acceptor absorption is sufficient to enable energy transfer from the donor to the acceptor as measured by a detectable increase in acceptor luminescence. Various coactivators for use in FRET assays have been described. Examples include, but are not limited to, Steroid Receptor Complex (SRC1), CREB binding protein (CBP), and Retinoid Interacting Protein (RIP 140). When a ligand binds to the ligand pocket of the receptor, the coactivator forms a receptor-coactivator complex. The current model on coactivators is that a ligand binds to the ligand binding domain (LBD) causing the activation function 2 (AF-2) to fold into place and trapping the ligand in the pocket. A novel interface (LXXLL motif) is formed by entrapment of the ligand, allowing the coactivator to interact with the AF-2. Thus, AF-2 is important in ligand dependent transactivation. When an agonist binds, it is transcriptionally active, while the binding of an antagonist interrupts the receptor cofactor interaction.

In the ERRγ FRET assay of the present invention RIP140(372-392) served as the coactivator. RIP140 was selected because both mammalian two-hybrid and Biacose experiments indicated a nanomolar interaction between ERRγ and RIP140. However, as will be understood by those of skill in the art upon reading this disclosure, other coactivators exhibiting similar interactions with ERRγ can also be used in identifying affinity ligands. The assay was set up in a one to one ratio of receptor to coactivator. However, as will also be understood by those of skill in the art upon reading this disclosure, other ratios can be used. A homology model was also develped from the ERα backbone with sequence analysis of ERRγ. This model showed the phenol binding site of ERs to be retained in the ERRs, thus indicating that a compound containing a phenol could serve as a ligand for ERRγ.

Further, while running this ligand sensing assay (LiSA) compounds that caused the receptor:cofactor interaction to disassociate were identified. Specifically, it was found that tamoxifen and 4-hydroxytamoxifen caused this dissociation. These data are indicative of thease compounds binding directly to the ERRγ:RIP140 complex, presumably through the ERRγ ligand binding site. Accordingly, a binding assay was also developed to determine if compounds were actually binding at the binding site or disrupting the LiSA in a different manner.

The binding assay was developed with a different set of parameters than that of the LiSA assay. No common set of assay conditions is applicable to all nuclear receptors. Accordingly, parameters to be optimized included pH, detergents, carriers, and bead concentrations. By clinging several variables at one time, a large number of conditions can be examined through the statistical capabilities of a program called JMP (SAS Institute, Cary N.C.). Multiple variables including, but not limited to, buffer, detergents, and carrier, were entered simultaneously Lnto the program with the goal being to increase specific counts while lowering non-specific counts. The parameters that were suggested by the program, namely MOPS, pH7, Tween 20, no carrier and 0.125 mg/ml beads were run in the binding assay. In this assay, binding was determined via competition between ³H tamoxifen and unlabeled 4-hydroxytamoxifen and/or tamoxifen.

Using the FRET assay and direct binding assay in conjunction with the homology model, it was found that analogs of tamoxifen (see Formula 1) are ligands for ERRγ. In particular, it was found that tamoxifen (see Formula 1), 3-hydroxytamoxifen (see Formula 2) and 4-hydroxytamoxifen (see Formula 3), of which the most preferred analog is 4-hydroxytamoxifen (see Formula 3), are ligands for ERPγ

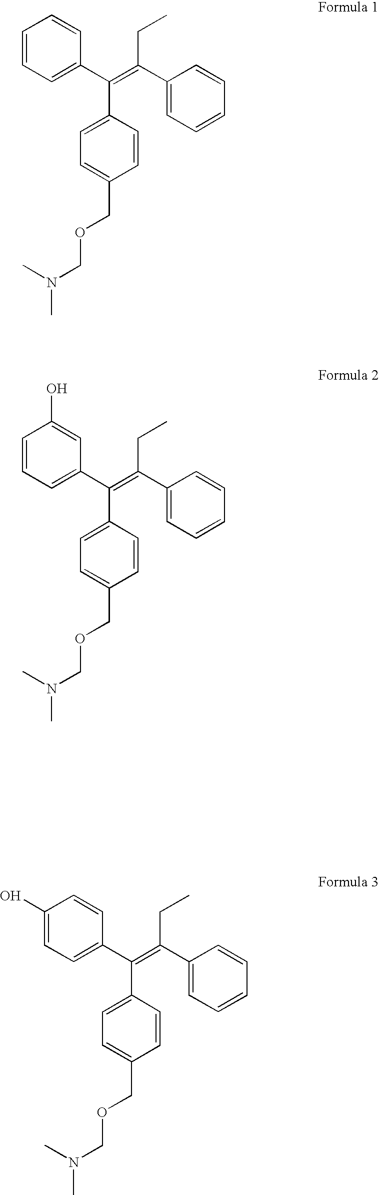

Other suitable compounds for screening in these assays include, but are not limited to, those derived from combinatorial libraries, defined chemical entities and compounds, peptides and peptide mimetics, oligonucleotide and natural product libraries, such as display (e.g. page display libraries) and antibody products. In a preferred embodiment, the test substances comprise organic molecules, preferably small organic molecules that have a molecular weight of from 50 to 2500 daltons. Candidate compounds may comprise biomolecules including saccharides, fatty acids, steroids, purines, pyrimidines, derivative and structural analogs or combinations thereof. These compounds are obtained from a wide variety of sources including libraries of synthetic or natural compounds. In addition, known pharmacological agents may be subjected to directed at random chemical modifications, such as acylation, alkylation, esterification, amidification, etc. to produce structural analogs for screening.

Compounds may be used in an initial screen of, for example, 10 different compounds per reaction. Those compounds showing inhibition or activation are then tested individually. Compounds may be used at a concentration of from 1 nM to 1000 μM, preferably from 1 μM to 100 μM more preferably from 1 μM to 10 μM. In a preferred embodiment, the activity of the compound is compared to activity shown by a known activator or inhibitor of ERRγ. A compound which acts as an inhibitor may produce a 50% inhibition of activity of ERRγ. Alternatively, a compound which acts as an activator may produce 50% of the maximal activity produced using a known activator of ERRγ.

Compounds identified as inhibitors or activators of ERRγ are believed to be useful in modulating the activity of this nuclear receptor. Accordingly, these compounds can be used in the prevention and/or treatment of ERRγ- and ER-mediated risk factors, diseases and conditions wherein activation or inhibition of ERRγ or ER is desired. Phenol containing compounds such as tamoxifen and analogs thereof such as 3-hydroxytamoxifen and 4-hydroxytamoxifen, as well as other compounds identified via the ERRγ:RIP140 FRET assay and/or the ERRγ direct binding assay can be administered in a therapeutically effective amount of the compound, preferably as a pharmaceutically acceptable composition, to treat ERRγ- and ER-mediated diseases, risk factors and conditions including, but not limited to osteoporosis cancer, menopause and cardiovascular disease. Accordingly, the present invention also relates to pharmaceutically acceptable compositions comprising a compound identified as an ERRγ ligand with one or more pharmaceutically acceptable carriers or excipients. In this aspect of the present invention only, it is preferred that the pharmaceutical composition comprise a compound other than tamoxifen or 4-hydroxytamoxifen. The carrier(s) or excipient(s) must be acceptable in the sense of being compatible with the other ingredients of the formulation and not deleterious to the recipient thereof.

Compounds of the present invention identified as ERRγ ligands may be formulated for administration by any route appropriate for the ERRγ- or ER-mediated disease to be treated. Suitable pharmaceutical formulations include, but are not limited to, those for oral, rectal, nasal, topical (including buccal and sublingual), vaginal or parenteral (including intramuscular, subcutaneous, intravenous, and directly into the affected tissue) administration or in a form suitable for administration by inhalation or insufflation. The formulation may, where appropriate be presented in convenient, discrete dosage units and may be prepared by any method well known in the art of pharmacy. All methods include the step of bringing into association the active ERRγ ligand with a liquid or finely divided solid carrier or both and then, if needed, shaping of the product into the desired formulation.

Pharmaceutical formulations suitable for oral administration may be presented in convenient discrete units including, but not limited to, capsules, cachews, or tablets, each containing a predetermined amount of the ERRγ ligand; as a powder or granules; as a solution, a suspension or as an emulsion. The ERRγ ligand can also be presented as a bolus, electuary, or paste. Tablets and capsules for oral administration may contain conventional excipients such as binding agents, fillers, lubricants, disintegrants, or wetting agents. The tablets may be coated according to methods well known in the art. Limed release formulations, which are known in the art, may also be suitable. Oral liquid preparations may be in the form of, for example, aqueous or oily suspensions, solutions, emulsions, syrups or elixirs, or may be presented as a dry product for constitution with water or other suitable vehicles before use. Such liquid preparations may contain conventional additives such as suspending agents, non-aqueous vehicles, including edible oils, or preservatives.

ERRγ ligands of the present invention may also be formulated for parenteral administration, such as by injection, for example bolus injection or continuous infusion, and may be presented in unit dose form in ampules, pre-filled syringes, small volume infusion or in multi-dose containers with an added preservative. Pharmaceutically acceptable compositions comprising an ERRγ ligand for parenteral administration may take the form of suspension, solution or emulsion in oily or aqueous vehicles, and may contain formulatory agents such as suspending, stabilizing, and/or dispersing agents. Alternatively, the active ingredient may be in powder form, obtained by asceptic isolation of sterile solid or by lyophilization from solution, for constitution with a suitable vehicle such as sterile, pyrogen free water before use.

For topical administration to the epidermis, ERRγ ligands of the present invention may be formulated as ointments, creams, or lotions, or as a transdermal peach. Ointments and creams, may, for example, be formulated with an aqueous or oily base with the addition of suitable thickening and/or gelling agents. Lotions may be formulated with an aqueous or oily base and will in general also contain one or more emulsifying agents, stabilizing agents, suspending agents, thickening agents, or coloring agents. Formulations suitable for topical administration in the mouth include lozenges comprising an ERRγ ligand in a flavored base, usually sucrose and acacia or tragacanth; pastilles comprising the active ingredient in an inert base such as gelatin and glycerin or sucrose and acacia; and mouth washes comprising the active ingredient in a suitable liquid carrier. For topical administration to the eye, the ERRγ ligands of the present invention can be made up in solution or suspension in a suitable sterile aqueous or non-aqueous vehicle. Additives such as buffers (e.g. sodium metabisulphite or disodium edeate) and thickening agents such as hypromellose can also be included.

Pharmaceutical formulations suitable for rectal administration wherein the carrier is a solid are preferably presented as unit dose suppositories. Suitable carriers include cocoa butter and other materials commonly used in the art, and the suppositories may be conveniently formed by admixture of the ERRγ ligand with the softened or melted carrier or carriers followed by chilling and shaping in molds.

Formulations suitable for vaginal administration may be presented as pessaries, tampons, creams, gels, pastes, foams, or sprays containing in addition to the ERRγ ligand such carriers as are known in the art to be appropriate.

For intra-nasal administration, ERRγ ligands of the present invention can be used as a liquid spray or dispersible powder or in the form of drops. Drops may be formulated with an aqueous or non-aqueous base also comprising one or more dispersing agents, solubilizing agents, or suspending agents. Liquid sprays are conveniently delivered from pressurized packs.

For administration by inhalation, ERRγ ligands of the present invention can be delivered by insufflator, nebulizer or a pressurized pack or other convenient means of delivering the aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount.

Alternatively, for administration by inhalation or insufflation, the ERRγ ligands of the present invention can take the form of a dry powder composition, for example a powder mix of an ERRγ ligand and a suitable powder base such as lactose or starch. The powder composition may be presented in unit dosage form in, for example, capsules, cartridges or blister packs of gelatins, from which the powder can be administered with the aid of an inhalator or insufflator.

When desired, any of the above-described formulations may be adapted to provide sustained release of the ERRγ ligand.

The pharmaceutical compositions of the present invention comprising an ERRγ ligand can also used in combination with other therapeutic agents.

The amount of an ERRγ ligand of the present invention required for use in treatment will of course vary not only with the particular ERRγ ligand selected but also with the route of administration, the nature of the condition being treated, and the age and condition of the patients. Selection of such an amount, referred to herein as the “therapeutically effective amount or concentration” is ultimately at the discretion of the attending physician. In general, however, suitable doses of pharmaceutical compositions of the present invention providing a therapeutically effective amount of an ERRγ ligand will be in the range of from about 0.1 to 300 mg/kg of bodyweight per day, particularly from about 1 to 100 mg/kg of bodyweight per day. An appropriate dosage unit for oral administration generally contains from about 1 to about 250 mg, more preferably 25 to 250 mg of an ERRγ ligand.

For use in the treatment of ERRγ- and ER-mediated diseases, risk factors and conditions including, but: not limited to osteoporosis, cancer, menopause and cardiovascular disease, pharmaceutical compositions comprising an ERRγ ligand can be administered by any of the aforementioned routes, preferably by the oral route or by injection. The daily dosage for a 70 kg mammal will typically be in the range of about 5 mg to 5 grams of an ERRγ ligand of the present invention.

The following nonlimiting examples are provided to further illustrate the present invention.

### EXAMPLES

### Example 1

### Protein Expression

Human Estrogen Related Receptor γ Ligand Binding Domain (ERRγ LBD) was expressed in E. coli strain BL21(DE3) is an amino-terminal poly-histidine tagged fusion protein. Sequence encoding a modified polyhistidine tag (MKKGHHHHHHG (SEQ ID NO:1) was fused in frame to a sequence encoding residues 229-458 of ERRγ and subcloned into the pRSETa expression vector (Invitrogen). The coding sequence of ERRγ LBD was derived from GenBank(AF094518). The resulting complete encoded sequence which is inclusive of the modified polyhistidine tag and ligand binding domain, SEQ ID NO:2, is as follows:

MKKGHHHHHH GPAKKPYNKI VSHLLVAEPE KIYAMPDPTV
PDSDIKALTT LCDLADRELV VIIGWAKHIP GFSTLSLADQ
MSLLQSAWME ILILGVVYRS LSFEDELVYA DDYIMDEDQS
KLAGLLDLNN AILQLVKKYK SMKLEKEEFV TLKAIALANS
DSMHIEDVEA VQKLQDVLHE ALQDYEAGQH MEDPRRAGKM
LMTLPLLRQT STKAVQHFYN IKLEGKVPMH KLFLEMLEAK V

Cells were grown in twelve one-liter flasks at 25° C. in LB medium with 0.1 mg/mL Carbenicillin for approximately 20 hours. Cells were harvested by centrifugation and concentrated cell slurries were stored in TBS, pH 8.0 at −80° C.

### Example 2

### Protein Purification

Approximately 70 grams wet cell paste was resuspended in 600 mL TBS, pH 8.0 (25mM Tris, 150 mM NaCl). Cells were lysed by passing 3 times through an APV Rannie MINI-lab homogenizer and cell debris was removed by centrifugation (45 minutes, 20,000 g, 4° C.). The cleared supernatant was filtered through coarse pre-filters, and TBS, pH 8.0 (containing 500 mM imidazole) was added to obtain a final imidazole concentration of 50mM. This lysate was loaded onto a column (XK-26, 10 cm) packed with Sepharose [Ns⁺ charged] Chelation resin (Pharmacia) and pre-equilibrated with TBS pH 8.0/50 mM imidazole. After washing to baseline absorbance with equilibration buffer, the column was washed with approximately one column volume of TBS, pH 8.0 (containing 95 mM imidazole). ERRγ LBD (229-458) was deluted with a linear gradient of 50 to 500 mM imidazole in TBS pH 8.0. Column peak fractions were pooled and immediately diluted 5 fold with 25 mM Tris, pH 8.0 (containing 5% 1,2-propanediol, 5 mM DTT and 0.5 mM EDTA). The diluted protein sample was then loaded onto a column (XK-16, 10cm ) packed with Poros HQ resin. After washing to baseline abscorbance with the dilution buffer, the protein was eluted with a gradient from 0-500 mM NaCl. Peak fractions were pooled and concentrated using Centri-prep 10K (Amicon) filter devices and subjected to size exclusion chromatograpy using a column (XK-26, 90 cm) packed with Superdex 75 column (Pharmacia) pre-equilibrated with TBS, pH 8.0 (containing 5% 1,2-propanediol, 5 mM DTT and 0.5 mM EDTA).

### Example 3

### Protein Biotinylation

Purified ERRγ LBD was buffer exchanged using PBS-10 gel filtration columns into PBS [100 mM NaPhosphate, pH 8.0, 150 mM NaCl]. ERRγ LBD was diluted to approximately 51 μM in PBS and five-fold molar excess of NHS-LC-Biotin (Pierce) was added in a minimal volume of PBS. This solution was incubated with gentle mixing for 2 hours at ambient room temperature. The biotinylation modification reaction was stopped by the addition of 2000× molar excess of Tris-HCl, pH 8.0. The modified ERRγ LBD was dialyzed against 3 buffer changes, each of at least 50 volumes; TBS, pH 8.0 (containing 5 mM DTT, 2 mM EDTA and 2% sucrose). This modified protein was distributed into aliquots, frozen on dry ice and stored at −80° C. The biotinylated ERRγ LBD was subjected to mass spectrometric analysis to reveal the extent of modification by the biotinylation reagent, In general, approximately 95% of the protein had at least a single site of biotinylation; and the overall extent of biotinylation followed a normal distribution of multiple sites, ranging from one to five.

### Example 4

### LiSA

Protein-protein interactions were assayed by FRET detection. Proteins were set up in a one to one ratio of Europium labeled streptavidin (Wallac CR28-100)-ERRγ APC labeled streptavidin (Molecular Probes #S-868) -RIP140 Complex. The RIP140 peptide (LCD2, 373-392) B-LERNNIKQAANNSLLLHLLKSQTIP-CONH₂ (SEQ ID NO:3) was prepared by SynPep. In this sequence, B represents biotin, CONH₂ indicates an amidated C-terminus and the other letters refer to the standard one letter amino acid code. The buffer for this system was made at 50 mM Hepes (pH 7), 50 mM KCl, 1 mM EDTA, in 1 L of deionized water. Thiii buffer was then filtered with the Corning (431205) filter system with a 0.22 μm cellulose Acetate filter. After filtering there was an addition of 0.1 mg/mL BSA (fatty acid free), and 2 mM Chaps. Before using the buffer in the assay, 10 mM DTT was added to the appropriate amount of buffer. The proteins were incubated for 30 minutes then excess biotin was added to fill vacant streptavidin (SA) sites. Protein mixture was added to prepared plates. They were then counted on the Wallac, Victor, and counts were then analyzed.

### Example 5

### Binding Assay

Binding assays were performed with [³H]-Tamoxifen (Amersham) . The same ERRγ protein as set forth in Example 4 was used to develop the binding assay. The buffer for this system was prepared with 50 mM MOPS (pH 7), 50 mM KCl, and 1 mM EDTA, in 1 L of deionized water. This buffer was then filtered with the Corning (431205) filter system with a 0.22 μm cellulose Acetate filter. After filtering, 0.01% Tween20 was added. Before using the buffer in the assay, 10 mM DTT was added to the appropriate amount of buffer. Approximately 0.125 mg/ml SA-SPA beads (Wallac) were coated with 20 nM biotinylated ERRγ. These beads were added to plates that had 100 nM ³H Tamoxifen, and 10 μM compounds of interest. These plates were then read on the Wallac Trilux scintillation counter.

1. A method of identifying compounds that will be useful for treatment of ERRγ- and ER-mediated diseases, risk factors, and conditions comprising the step of determining whether the compound interacts with ERRγ.

2. The method of claim 1 wherein the identified compound will be useful for the treatment of osteoporosis, cancer, menopause, or cardiovascular disease.

3. The method of claim 1 wherein the step of determining whether the compound interacts with ERRγ comprises an ERRγ:coactivator FRET assay or ERRγ direct binding assay.

4. The method of claim 3 wherein the coactivator of the FRET assay is RIP140.

5. A method for treating an ERRγ or ER-mediated disease, risk factor, or condition comprising administering a therapeutically effective amount of a compound identified by the method of claim 1.

6. The method of claim 5 wherein the ERRγ- or ER-mediated disease, risk factor or condition comprises osteoporosis, cancer, menopause, or cardiovascular disease.

7. The method of claim 5 wherein the compound comprises a phenol group.

8. The method of claim 5 wherein the compound is tamoxifen, 3-hydroxytamoxifen, or 4-hydroxytamoxifen.

9. The method of claim 5 wherein the compound is 4-hydroxytamoxifen.

10. A pharmaceutical composition comprising a compound identified to interact with ERRγ in accordance with the method of claim 1 and one or more pharmaceutically acceptable carriers or excipients.

11. The pharmaceutical composition of claim 10 wherein the compound is not tamoxifen or 4-hydroxytamoxifen.
